(19) United States
(12) Patent Application Publication
(10) Pub. No.: US 2003/0104357 A1
(43) Pub. Date: Jun. 5, 2003

(54) Jaagsiekte sheep retroviral packaging cell lines and methods relating thereto

(75) Inventors: Sharath K. Rai, Waterford, CT (US); Arthur Dusty Miller, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Seattle, WA (US)

(21) Appl. No.: 10/112,178

(22) Filed: Mar. 29, 2002

Related U.S. Application Data
(60) Provisional application No. 60/280,515, filed on Mar. 29, 2001.

(51) Int. Cl. C12Q 1/70; C12P 21/06; C12P 19/34; A61K 39/12; A61K 39/21

(52) U.S. Cl. 435/5; 424/208.1; 435/91.1; 435/69.1; 424/204.1

(57) ABSTRACT

The present invention demonstrates that JSRV envelope protein (Env) can be used to transduce human and other mammalian cells. Hybrid retrovirus packaging cells have been constructed that express the JSRV Env and retrovirus Gag-Pol proteins, and can produce JSRV-pseudotype vectors at high titers. Using high-titer virus the host range for JSRV has been established, and included sheep, human, monkey, bovine and dog cells, but not murine, rat or hamster cells. Retroviral packaging cell lines comprising the JSRV envelope protein, and receptor binding fragments thereof, are provided which transiently and stably produce high titers of recombinant retrovirus particles which can be used to transfer a heterologous gene to a eukaryotic cell.

### CORRESPONDING RELATED APPLICATIONS

This application is a continuation in part of and claims priority to U.S. Ser. No. 60/280,515, filed Mar. 29, 2001, the disclosure of which is incorporated herein by reference in its entirety.

### STATEMENT AS TO RIGHTS TO INVENTIONS MADE UNDER FEDERALLY SPONSORED RESEARCH AND DEVELOPMENT

This work was supported by grants DK47754, HL54881, and CA59116 from the National Institutes of Health. The U.S. Government may have certain rights in the invention.

### BACKGROUND OF THE INVENTION

Jaagsiekte sheep retrovirus (JSRV) is the causative agent of a contagious lung cancer of sheep known as ovine pulmonary carcinoma (OPC), also known as sheep pulmonary adenomatosis (SPA) or jaagsiekte. OPC is a veterinary problem with significant economic impact in several countries. In addition, OPC shares characteristics with human bronchioalveolar carcinoma (BAC) (Clayton et al., Pathol. Annu. 23:361-394 (1988); Ives et al., Am. Rev. Respir. Dis. 128:195-209 (1983); Perk et al., J. Nat. Cancer Inst. 69:747-749 (1982)), and BAC represents about 25% of human lung cancer cases (Barsky et al., Cancer 73:1163-1170 (1994)). Lung cancer being the most common fatal form of cancer in humans (Carney et al., Semin. Oncol. 15:199-214 (1988)), recent interest in JSRV stems from the hypothesis that OPC could be useful as a naturally occurring animal model for understanding the mechanism of pulmonary carcinogenesis (Hecht et al., Br. Vet. J. 152:395-409 (1996); Perk et al., J. Nat. Cancer Inst. 69:747-749 (1982)).

JSRV has been classified as a type D retrovirus, based on genomic organization, but has a type B-like envelope (Env) protein (York et al., J. Virol. 66:4930-4939 (1992)). The sheep genome carries multiple copies of JSRV-like endogenous sheep retrovirus (ESRV) sequences (Hecht et al., Virology 202:480-484 (1994); Hecht et al., Proc. Natl. Acad. Sci. USA. 93:3297-3302 (1996); York et al., J. Virol. 66:4930-4939 (1992)), but subsequent studies have shown that JSRV is an exogenous virus distinct from ESRV sequences (Bai et al., Virology 258:333-343 (1999); Bai et al., J. Virol. 70:3159-3168 (1996); Palmarini et al., J. Virol. 70:1618-1623 (1996)) and is specifically associated with OPC. Recent studies by Palmarini et al. (J. Virol. 73:6964-6972 (1999)) using an infectious molecular clone of JSRV have confirmed that JSRV is the causative agent of OPC. The mechanism of oncogenesis by JSRV is not known. JSRV has the genomic organization of a simple replication-competent retrovirus with no known oncogenes. The incubation period in naturally acquired OPC seems to range from months to years, suggesting insertional mutagenesis. However, OPC can be induced experimentally in 3 to 4 weeks, suggesting a mechanism of action more similar to a transforming retrovirus.

JSRV has recently gained prominence mainly because of the similarity of OPC to BAC in humans, suggesting OPC can be used as an animal model to understand the process of pulmonary carcinogenesis. However, studies on JSRV have been hindered by the lack of a cell culture system for propagating the virus. Recently a full length infectious proviral molecular clone of JSRV has been isolated from a natural case of OPC (Palmarini et al., J. Virol. 73:6964-6972 (1999)). JSRV has been shown to infect sheep and goats, but there is no evidence of human infection.

The present invention demonstrating the ability of JSRV Envelope protein to recognize and infect mammalian cells, including sheep, human, monkey, bovine, dog and rabbit cells, the making of packaging cells and cell lines, and replication-defective hybrid retroviral particles which bind the human JSRV receptor provide additional needed compositions and methods for the transfer of genes to cells, particularly human cells. The methods and compositions of the present invention are particularly useful for the transfer of genes to cells of the human lung epithelium. Additional needs well known in the art are fulfilled by the present invention.

### SUMMARY OF THE INVENTION

In one aspect the invention provides a cultured packaging cell for producing a replication-defective retroviral vector particle. The packaging cell is a vertebrate cell capable of expressing and assembling retroviral proteins, and comprises a first expression vector encoding a retroviral envelope (Env) protein having amino acid residues of the Jaagsiekte sheep retrovirus (JSRV) envelope that directs binding of the replication-defective retroviral particle to a JSRV retroviral receptor on a target cell. The packaging cell further comprises a second vector encoding a retroviral Gag and a Pol protein, such that upon expression of the first and second vectors in the presence of a retroviral vector having a nucleotide sequence capable of encoding a heterologous gene of interest, a replication-defective retroviral vector particle is produced that binds to JSRV retroviral receptors of target cells. In one particular embodiment the packaging cell can produce the replication-defective retroviral particle transiently, while in additional embodiments the packaging cell can be a stable cell line.

Retroviral gag and pol genes for use in the present invention can be from, for example, amphotropic and xenotropic retroviruses. Typically, the gag and pol genes can be derived from retroviruses including type B, mammalian and avian type C, and type D retroviruses. For example, the gag and pol genes can be from, but not limited to, oncoretroviruses including, but not limited to, type B retroviruses such as murine mammary tumor virus (MMV), mammalian and avian type C retroviruses such as rous sarcoma virus (RSV), gibbon-ape leukemia virus (GALV), feline leukemia virus (FLV), Moloney murine leukemia virus (MoMLV), and type D retroviruses such as Jaagsiekte sheep retrovirus (JSRV), simian retrovirus (SRV), squirrel Monkey retrovirus (SMRV), enzootic nasal tumor virus (ENTV), and Simian Mason-Pfizer virus (MPMV) or lentivirus including, but not limited to, human immunodeficiency virus (HIV), simian immunodeficiency virus (SIV), equine infectious anemia virus (EIAV), feline immunodeficiency virus (FIV), bovine immunodeficiency virus (BIV), caprine arthritis encephalitis virus (CAEV) and Visna lentivirus (VILV).

The cultured packaging cell can be an avian or mammalian cell capable of expressing and assembling retroviral proteins. In one embodiment of the present invention the cultured packaging cell produces the replication-defective retrovirus transiently. While in another embodiment, the vectors encoding the retroviral Env protein, the retroviral Gag and Pol proteins, and the nucleotide sequence encoding a heterologous gene of interest can be integrated in a chromosome of the packaging cell forming a stable cell line.

In another aspect, the present invention provides a packaging cell comprising an expression vector encoding the receptor binding region of JSRV Env protein. In a particular embodiment, the receptor binding region of JSRV ENV protein comprises the amino- (NH₂—) terminal amino acid residues of JSRV envelope protein and the hinge and carboxyl-terminal amino acid residues (i.e., comprising the membrane fusion domain) of another retrovirus to form a chimeric envelope protein. In a particular embodiment the chimeric envelope protein comprises the signal sequence, the receptor binding domain and the transmembrane domain, including the membrane-spanning domain of JSRV and the remaining amino acid residues corresponding to the cytoplasmic domain of another retrovirus. For example the cytoplasmic domain can be from a lentivirus or an oncoretrovirus including for example, but not limited to, the cytoplasmic domain of Moloney murine leukemia virus envelope protein, human immunodeficiency virus or simian immunodeficiency virus envelope proteins.

In yet another embodiment, the present invention provides JSRV Env protein having a mutation in the cytoplasmic domain at Tyr 590 (SEQ ID NO.: 2 and SEQ ID NO: 4) or at Met 593 (SEQ ID NO.: 2 and SEQ ID NO.: 4). In particular the mutation at Tyr 590 can be a replacement with Phe, Cys or Asp and the mutation at Met 593 can be a replacement with Glu. Mutations at these positions can result in the lose of any transformation activity that might be present if the native or wild type cytoplasmic domain of JSRV Env protein is used.

In still another aspect the invention provides a method for producing a replication-defective retroviral vector particle encoding a heterologous protein of interest. The method comprises transducing or transfecting a retroviral packaging cell with (i) a replication-defective retrovirus particle which comprises virus RNA transcribed from a recombinant DNA provirus, the provirus comprising virus long terminal repeat sequences (LTRs), a retrovirus packaging sequence, and a heterologous gene, or (ii) a vector comprising the provirus. The packaging cell can be a vertebrate cell capable of expressing and assembling retroviral proteins and having (i) an integrated expression vector encoding a retroviral Env protein having amino acid residues of the JSRV virus (JSRV) that direct binding of the retroviral particle to the JSRV retroviral receptors on a target cell, and (ii) an integrated vector encoding retroviral Gag and Pol proteins.

In one embodiment the Gag and Pol proteins can be from a retrovirus other than JSRV thereby producing a replication-defective hybrid retroviral particle. As above, the Gag and Pol proteins can be from, but not limited to, a oncoretrovirus of type B, avian and mammalian type C, or type D, or a lentivirus. The nucleotide sequences which encode the retroviral Env protein, the retroviral Gag and Pol proteins, and the nucleotide sequence that encodes the heterologous protein of interest are expressed, producing a replication-defective hybrid retroviral vector particle that binds to JSRV retroviral receptors of target cells. In another aspect the invention provides a replication-defective retroviral vector particle containing the heterologous gene of interest produced by this method.

### DESCRIPTION OF THE SPECIFIC EMBODIMENTS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

The practice of the present invention employs, unless otherwise indicated, conventional techniques of molecular biology, microbiology, recombinant DNA, and immunology, which are within the skill in the art. Such techniques are described fully in the literature. See, for example, Sambrook et al., Molecular Cloning: A Laboratory Manual, Second Edition (1989), Oligonucleotide Synthesis Gait, ed., (1994), Animal Cell Culture, Freshney, ed., (1987), the series Methods in Enzymology (Academic Press, Inc.); Gene Transfer Vectors for Mammalian Cells, Miller and Carlos, eds. (1987); Handbook of Experimental Immunology, 4th Edition, Weir et al., eds., (1986); Current Protocols in Molecular Biology, Ausubel et al. eds. (1987); Current Protocols in Immunology, Coligan et al. eds. (1991).

Definitions

The terms “polypeptide”, “peptides” and “protein” are used interchangeably to refer to polymers of amino acids of any length. These terms also include proteins that are post-translationally modified through reactions that include, but are not limited to, glycosylation, acetylation and phosphorylation.

“Polynucleotide” refers to a polymeric form of a nucleic acid of any length, either ribonucleotides or deoxyribonucleotides, or analogs thereof. This term refers only to the primary structure of the molecule, the sequence of nucleotides. Thus, double- and single-stranded DNA, as well as double- and single-stranded RNA are included. It also includes modified polynucleotides such as methylated or capped polynucleotides.

A “gene” refers to a polynucleotide, or polynucleotide sequence, containing at least one open reading frame that is capable of encoding a particular protein after being transcribed and translated.

“Operably linked” or “operably associated” refers to an arrangement of two or more components, wherein the components described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to the coding sequence.

“Recombinant” refers to a genetic entity distinct from that generally found in nature. As applied to a polynucleotide or gene, this means that the polynucleotide, or polynucleotide sequence, is the product of combinations of molecular manipulations including, but not limited to, cloning, restriction and/or ligation steps, and other procedures that result in a construct that is distinct from the polynucleotide found in nature.

“Heterologous” means derived from a genotypically distinct entity from that of the rest of the entity to which it is compared. For example, a polynucleotide introduced by genetic engineering techniques into a different cell type is a heterologous polynucleotide. When that polynucleotide is expressed, the polynucleotide can encode a heterologous polypeptide. Similarly, a LTR or promoter that is removed from its native coding sequence and operably linked to a different coding sequence is a heterologous LTR or promoter.

“Packaging” as used herein refers to a series of subcellular events that results in the assembly and encapsidation of a viral vector, particularly a retrovirus vector. Thus, when a suitable vector is introduced into a packaging cell under appropriate conditions it can be assembled into a viral particle. Functions associated with packaging of viral vectors, particularly retroviral vectors, are described herein and in the art.

“Transduce”, or “transfect” as used herein refers to the transfer of non-viral DNA to a cell, i.e., a bacterial or eukaryotic cell.

A “host cell”, “cell line”, “cell culture”, “packaging cell” and other such terms describe higher eukaryotic cells, preferably mammalian cells, most preferably human cells, useful in the present invention. These cells can be used as recipients for recombinant vectors, viruses or other transfer polynucleotides, and include the progeny of the original cell that was transduced. It is understood that the progeny of a single cell may not necessarily be completely identical, in, for example, morphology or in genomic complement, to the original parental cell.

A “therapeutic gene”, “target polynucleotide”, “transgene”, “gene of interest” and the like, generally refer to a gene or genes to be transferred using a recombinant virus vector, i.e., a heterologous gene. Typically, in the context of the present invention, such genes are located within the recombinant replication-defective virus vector, which vector is flanked by retroviral long terminal repeat (LTR) regions and therefore can be replicated and encapsidated into recombinant retroviral particles including hybrid retroviral particles.

A “hybrid” replication-defective retroviral particle as used herein refers to a retroviral particle comprising Env, Gag and/or Pol proteins from other different retroviruses. Retroviruses from which Gag and Pol proteins useful in the present invention can be derived from include, but are not limited to, type B, mammalian and avian type C, and type D retroviruses. For example, the gag and pol genes encoding the Gag and Pol proteins of the retroviral particles can be from, but not limited to, oncoretroviruses including, but not limited to, type B retroviruses such as murine mammary tumor virus (MMV), mammalian and avian type C retroviruses such as rous sarcoma virus (RSV), gibbon-ape leukemia virus (GALV), feline leukemia virus (FLV), Moloney murine leukemia virus (MoMLV), and type D retroviruses such as Jaagsiekte sheep retrovirus (JSRV), simian retrovirus (SRV), squirrel Monkey retrovirus (SMRV), enzootic nasal tumor virus (ENTV), and Simian Mason-Pfizer virus (MPMV) or lentivirus including, but not limited to, human immunodeficiency virus (HIV), simian immunodeficiency virus (SIV), equine infectious anemia virus (EIAV), feline immunodeficiency virus (FIV), bovine immunodeficiency virus (BIV), caprine arthritis encephalitis virus (CAEV) and Visna lentivirus (VILV). For example, the Env protein of a specific embodiment of the present invention is the JSRV Env protein and the Gag and Pol proteins are from the Moloney murine leukemia virus.

To effect expression of the transgene in a recipient host cell, it is generally operably linked to a promoter, either its own or a heterologous promoter. A large number of suitable promoters are known in the art, the choice of which depends on the desired level of expression of the transgene; whether one wants constitutive expression, inducible expression, cell-specific or tissue-specific expression, and the like. The recombinant retroviral vector can also contain a selectable marker.

In particular, the present invention provides retrovirus packaging cell lines based on the Jaagsiekte sheep retrovirus (JSRV) Env protein. Such vectors are capable of expressing replication-defective retroviral particles that are capable of using a JSRV receptor for cell entry. The Jaagsiekte sheep retrovirus (JSRV) is capable of infecting a variety of cell types across a wide host range including sheep, goat, human, monkey, bovine, dog and rabbit, but not mouse, rat or hamster cells. Thus, the JSRV receptor is present on a variety of cells rendering JSRV pseudotype packaging cells useful in methods of mammalian, and particularly human gene transfer.

The present invention also provides the first evidence JSRV Env can transduce, or transfer genes, to human cells and provides for feasible methods for producing a recombinant retroviral vector for the transfer of a heterologous gene to the cells of the lung epithelium. Methods using both viral (Flotte et al., Proc. Natl. Acad. Sci. USA. 90:10613-10617 (1993); Rosenfeld et al., Cell 68:143-155 (1992)) and non-viral (Alton et al., Nature Genet. 5:135-142 (1993); Hyde et al., Nature 362:250-255 (1993)) means have been extensively studied for gene transfer to airway epithelial cells. But, to date efficient gene transfer to the airway epithelial cells has proved difficult (Goldman et al., Hum. Gene Ther. 8:2261-2268 (1997); Grubb et al., Nature 371:802-806 (1994); Pickles et al., J. Virol. 72:6014-6023 (1998)).

Amphotropic retroviral vectors have been shown to efficiently transduce the basal and secretory airway epithelial cells in vitro, but in vivo delivery resulted in no detectable transduction in the intact normal airway epithelium and a low transduction rate in the wounded epithelium (Halbert et al., Hum. Gene Ther. 7:1871-1881 (1996)). Low retroviral transduction in vivo is apparently due to the low abundance of retroviral receptors and inhibition of amphotropic retroviral vector transduction by pulmonary surfactant (Zsengeller et al., Hum. Gene Ther. 10:341-353 (1999)) or by soluble chondroitin sulfates in pleural effusions (Batra et al., J. Biol. Chem. 272:11736-11743 (1997)). However, the retroviral vectors of the present invention bearing the JSRV Env have been demonstrated to be stable to treatment with lung surfactant unlike otherwise identical vectors bearing amphotropic Env which were inactivated. (Coil et al., J. Virol. 75:8864-8867 (2001).

JSRV has been shown to infect several cell types in vivo (Holland et al., J. Virol. 73:4004-4008 (1999); Palmarini et al., J. Gen. Virol. 76:2731-2737 (1995); Palmarini et al., J. Virol. 73:6964-6972 (1999)). The epithelial tumor cells in the lungs of sheep have been shown to be the major sites of viral replication (Palmarini et al., J. Gen. Virol. 76:2731 -2737 (1995)) suggesting a natural tropism of the virus for the airway epithelial cells. Further, intratracheal inoculation of concentrated lung fluid collected from OPC-affected sheep in new born lambs has experimentally reproduced OPC (DeMartini et al., J. Natl. Cancer Inst. 79:167-177 (1987); Martin et al., Nature 264:183-185 (1976); Verwoerd et al., Onderstepoort J. Vet. Res. 47:275-280 (1980)) demonstrating the stability of the virus in lung fluid. These characteristics provide for retroviral vectors as described below for use in efficiently transferring genes to human lung epithelial cells.

Structural studies on the envelope glycoprotein of the mammalian C-type retroviruses have demonstrated that the receptor-binding function is localized to the amino-(NH₂—) terminal region of the surface glycoprotein domain (Davey et al., J. Virol 71:8096-8102 (1997)). The receptor binding domain structure of the Friend murine leukemia virus (FR-MLV), for example, amino acid residues 1-236, has been examined at 2.0 Å resolution (Fass et al., Science 277:1662-1665 (1997)). Certain regions of the Env domain have been identified that vary with host tropism. These variable regions, designated VRA, VRB, and VRC vary in length as well as amino acid residue sequence between murine leukemia viruses with different host tropisms.

JSRV Env is a type B envelope based on deduced amino acid homologies (Hecht et al., Proc. Natl. Acad. Sci. USA 93:3297-3302 (1996) and York et al., J. Virol. 66:4930-4939 (1992)). The JSRV env encodes a precursor molecule of about 615 amino acid residues. The nucleotide sequences and amino acid sequences for various isolates of JSRV Env are provided in, for example, York et al. (J. Virol. 66:4930-4939 (1992) SEQ ID NOs.: 1 and 2) and Bai et al., (Virology 268:333-343 (1999), JS7 designated SEQ ID NOs: 3 and 4). The C terminus of the outer membrane protein, or surface protein (SU) and the NH₂ extremity of the transmembrane domain (TM) is defined by the protease cleavage sequence ArgProLysArg/GlyLeuSer (amino acid residues 375-381 of SEQ ID NO: 2). Three hydrophobic segments can be readily identified in the Env precursor. The first one, 13 amino acids long, begins at about amino acid residue 61 of SEQ ID NO: 2, downstream of the start site, corresponds to the signal sequence. The mature JSRV Env protein begins at approximately amino acid residue 77 of SEQ. ID. NO.: 2. The second is about 25 amino acid residues long and was located just downstream of the SU-TM cleavage site (beginning at about amino acid residue 382 of SEQ ID NO: 2), and corresponds to the fusiogenic domain, and the third, comprises about 24 amino acid residues and represents the anchor, membrane-spanning domain of TM (beginning at about amino acid residue 550 of SEQ ID NO: 2), which is followed by the cytoplasmic domain

In general, the retroviral envelope protein includes a surface (SU) domain, or surface protein, and a transmembrane (TM) domain or protein. The surface protein of, for example, an amphotropic envelope protein includes, in an N-terminal to C-terminal direction, the following regions: (1) a receptor binding region; (2) a hypervariable region; and (3) a body portion, which is associated with the transmembrane domain. The hypervariable region functions as a linker that connects the receptor binding region and the body portion of the protein. Retroviruses can be made targetable to a desired ligand by modifying the receptor binding region such that this region includes for example, a polypeptide which binds to a particular cell surface ligand.

Typically, the nucleotide sequence which encodes the signal sequence, extracellular domain and transmembrane domain, including the membrane-spanning portion, of JSRV Env (amino acid residues 1 through 571 as depicted in SEQ ID NO: 2) will be used with nucleotide sequences which encode a cytoplasmic domain of another amphotropic or xenotropic retrovirus Env comprising the remainder of the Env protein. The cytoplasmic domain from the Env of retroviruses including, but not limited to, MoMLV, HIV, and the like are suitable for this use. In additional embodiments the signal sequence can be removed without a lose of receptor specificity. Also, nucleotide sequences comprising a chimeric Env can include only the JSRV receptor binding region and the remaining envelope domains from other retrovirus envelope proteins, including the membrane fusion domain, transmembrane domain and cytoplasmic domain to encode a chimeric JSRV Env that can recognize and bind the JSRV receptor (JVR) maintaining the host range of JSRV.

Chimeric JSRV Env protein used to make a JSRV packaging cell of the present invention will typically comprise the NH₂-terminal encoding domain of the JSRV env gene (from about nucleotide 181 to about nucleotide 1134 of SEQ ID NO: 1) operatively associated with a retrovirus hinge and TM encoding domain of another retrovirus so as to express a chimeric protein which can bind with the JSRV receptor and to maintain the host range of JSRV. Further, it is contemplated as part of the present invention to insert JSRV receptor binding domains which functionally correspond to VRA, VRB and VRC domains of a type C retrovirus into a framework nucleotide sequence which encodes a second retrovirus surface protein to provide a retrovirus surface protein which binds to JSRV but shares the membrane hydrophobic fusion peptide and transmembrane segments of a second retrovirus. The chimeric surface protein can demonstrate a broader host range than JSRV when variable domains VRA, VRB and VRC are recombined from different retroviruses.

Further, it has been found that the expression of the JSRV Env is sufficient to transform mouse NIH 3T3 cells. (Palmarini et al., J. Virol. 75:11002-11009 (2001). Mutations at Tyr 590 and M 593 (SEQ ID NO.: 2 and 4) have been found to inhibit the transformation activity of the cytoplasmic domain of JSRV ENV. Therefore, nucleotide sequences encoding the entire JSRV with mutations at TYR 590 and M 593 can be used in the vectors of the present invention. In particular, the mutation at the nucleotide sequence that encodes Tyr 590 can be such to encode a replacement with Phe, Cys or Asp and the mutation at the nucleotide sequence that encodes Met 593 can be such to encode a replacement with Glu. Mutations at these positions result in the loss of any transformation activity that might be present if the native or wild type cytoplasmic domain of JSRV Env protein is used.

The JSRV packaging cells of the invention are a reproducible source of retroviral particles. JSRV packaging can produce retrovirus particles either transiently or stable cell lines can be isolated from populations that produce high titers of virus. The packaging cell lines of the invention can be selected and cloned for other desirable properties, such as stability of in vivo growth, lack of production of helper virus, lack of reinfection by viral particles packaged in the cell, stability from genetic rearrangement and recombinational events, resistance to complement lysis, and improved ability to infect cells from higher mammals. The replication defective virus vectors produced by the JSRV packaging cell lines of the present invention permit the transfer of a wide variety of heterologous nucleic acid segments. Heterologous nucleic acid segments encompass polynucleotide sequences (for example, DNA or RNA corresponding to the DNA) encoding proteins, polypeptides, or peptides of interest, and RNA molecules such as antisense RNA. Polynucleotides which encode a protein of interest include genes, cDNAs, DNA sequences from libraries,, i.e., a cDNA library, and minigenes.

In a particular embodiment, hybrid JSRV packaging cells that transiently produce replication defective particles comprising a chimeric JSRV Env having a receptor binding domain and transmembrane domain of JSRV and Gag and Pol from HIV Env have been made. These hybrid retroviral particles are of particular interest because a heterologous gene transferred by this particle can be expressed in non dividing cells. Stable packaging cell lines can be isolated from high expressing transient populations of cells because chimeric JSRV Env as used in the present invention are not toxic or tumorigenic. VSVG packaging lines have typically been used for producing hybrid retroviral particles with HIV Gag and Pol, but these lines can only produce these particles transiently because the VSVG Env is toxic to the cells.

For use in gene therapy, a recombinant retroviral vector will contain a polynucleotide sequence or other DNA which is desired to be transferred to the cells of the intended recipient. Such polynucleotides include, but are not limited to, (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators, such as cytokines; (v) polynucleotides that can make recipient cells susceptible to specific drugs, such as the herpes virus thymidine kinase gene, and the like; and (vi) polynucleotides for the treatment of various cancers. The enzyme or other protein may function within a cell, or may be secreted and circulate in the body, such as, for example, a hormone, blood factors, and the like. Genes which code for proteins whose levels do not have to be precisely controlled, and/or genes which cause disease by virtue of a single defect, are particularly suitable for insertion into a retroviral vector packaged by a JSRV packaging cell line of the present invention.

The packaging cell line is transduced or transfected with a replication defective virus vector, or a DNA construct having a strand corresponding to or complementary to a replication defective viral vector, containing the heterologous gene(s) of interest. Representative genes useful in the present invention include, among others, those which encode, for example, blood clotting factors, adenosine deaminase, interleukins, interferons, GM-CSF, G-CSF, erythropoietin and other cytokines, receptors, cystic fibrosis transmembrane conductance regulator (CFTR), tumor suppressors, antisense polynucleotides, i.e., antisense RNAs, vaccine antigens, and the like. The vector comprises a sequence capable of providing retroviral long terminal repeats (LTRs), a sequence required for reverse transcription, a retroviral packaging sequence, and the polynucleotide sequence of interest. A DNA construct includes the LTRs necessary for host cell genome incorporation and expression. Following synthesis the viral DNA is integrated into cellular DNA so that the ends of the LTRs are directly joined to cellular sequences to form a stable structure (e.g., the provirus). The vector component necessary for reverse transcription does not necessarily include sequence coding for reverse transcriptase, but rather, includes a replication initiation site and a polypurine tract. A packaging signal, typically specific for the retroviral vector of interest, is included in the vector. Donor and acceptor splice sites and internal ribosome entry sites (IRES) can also be present in a replication defective virus vector. The splice sites enable the expression of additional heterologous inserted nucleic acid sequences.

To minimize the chance of producing infectious viral particles once the retroviral vector DNA is integrated into host cellular DNA, the replication defective virus vector need not include one or more of the sequences corresponding to the viral genes gag, coding for the viral core proteins, pol, coding for the viral RNA-dependent DNA polymerase (reverse transcriptase and other enzymatic proteins), or env, coding for the viral envelope proteins. The sequences can be omitted or rendered defective by mutagenesis.

For gene transcription from the replication defective virus vectors packaged by a JSRV cell line of the present invention, the gene of interest may be linked to a heterologous promoter. The selection of a suitable promoter is well within the level of ordinary skill in the art. A wide variety of promoters have been described in the literature including both viral and cellular promoters. Viral promoters include the immediate early cytomegalovirus promoter (Boshart et al., Cell 41:521-530 (1985), incorporated herein by reference), the murine leukemia virus (MLV) LTR promoter (Weiss et al., RNA Tumor Viruses, Cold Spring Harbor Laboratory Press, Cold Spring Harbor, N.Y., p766 (1985), incorporated herein by reference) the SV40 promoter (Subramani et al., Mol. Cell. Biol. 1:854-864 (1981), incorporated herein by reference) and the like. Cellular promoters include but are not limited to the mouse metallothionien-1 promoter (Palmiter et al., U.S. Pat. No. 4,579,821, incorporated herein by reference). Alternative splicing can also be exploited to facilitate the expression of polycistronic genes in the vector. In this strategy, one of the proteins encoded by the vector is translated from the full length vector RNA while partial splicing from a splice donor site near the 5′ LTR to a splice acceptor just upstream of a downstream gene yields a transcript that encodes the downstream gene product. Because vector replication involves an RNA intermediate, construction of the vector containing inserted gene(s) should permit full-length transcription of vector genome.

The replication defective retrovirus vector particles packaged by the JSRV cell lines in accordance with the present invention will often contain at least one exogenous (heterologous) gene.

Selectable markers can also be included in the replication defective retroviral vectors packaged according to the present invention, for investigative or experimental purposes, or to provide a means to select for cells containing the replication defective retroviral vectors. These markers include the neomycin and hygromycin phosphotransferase genes that confer resistance to G418 and hygromycin, respectively. Other markers include the mutant mouse dihydrofolate reductase gene (dhfr) which confers resistance to methotrexate, the bacterial gpt gene which allows cells to grow in medium containing mycophenolic acid, xanthine, and aminopterin, the bacterial hisD gene which allows cells to grow in medium without histidine but containing histidinol, and the multidrug resistant gene (mdr) which confers resistance to a variety of drugs. These markers are dominant selectable markers and allow chemical selection of most cells expressing these genes. Suicide genes may also be contained within the vectors packaged by the JSRV packaging cell lines of the present invention. Such genes provide a means to selectively kill cells containing the retroviral RNA. For example, the tk gene (Culver et al., Science 256:1550-1552 (1992), incorporated herein by reference) can be used in combination with gancyclovir to selectively kill transduced cells.

The exogenous, or heterologous, gene for insertion in the vector can be an intronless cDNA copy of an mRNA encoding a gene product of interest. Large inserts can be placed in the replication defective retroviral vectors, but generally the gene(s) of interest and any attendant regulatory sequences should be no more than up to approximately 8 to 11 kb in size. The 5′ and 3′ noncoding regions of the cDNA can be trimmed to reduce the size of the insert and to remove potential polyadenylation signals that may occur in the 3′ end of cDNAs. The cDNA can be inserted in the same transcriptional orientation as the viral LTR. Antisense RNAs can be expressed by reversing the orientation of the cDNA with respect to a promoter. Other inserts include intronless “minigenes” which include normal genes from which the introns have been removed and those with truncated coding regions. Entire genes containing introns can also be inserted, and typically will be inserted in reverse orientation to prevent removal of the introns during vector replication.

Packaging cell lines of the present invention can be constructed and optimized using a variety of strategies. In general, such strategies are designed to reduce the chance of recombination between a helper construct (i.e., one or more constructs that provide trans-acting proteins required for production of replication defective retroviral vector particles) and the vector that may result in the production of helper virus. In this context “helper virus” means undesirable replication-competent retrovirus produced from the integrated proviral genome in some packaging cells by genetic recombination and repair of the defective retroviral vector proviral genome. Strategies include but are not limited to helper constructs in which the packaging signal(s) have been deleted, helper constructs in which the gag, pol and env genes are split into two or more separate transcriptional units, e.g., containing gag-pol and env, or helper constructs in which the gag-pol and env genes are split into two separate transcriptional units and which contain mutations (e.g., by insertions of linkers) and deletions in the gag-pol and env transcriptional units.

In addition, the 3′ LTRs in separate transcriptional units can be replaced with polyadenylation signals from SV40, thereby requiring an additional recombinational event to generate helper virus. Avoidance of homologous overlap between vector and helper virus sequences in the JSRV packaging cells decreases the chance of helper virus production. This can be accomplished by removing as much of the helper virus sequences from the vector as possible. Suitable helper constructs are cotransfected with the vector of the present invention thus providing the required trans-acting proteins and allowing virus particle production. Trans-acting proteins may also be provided by packaging cell lines that are designed to provide all viral proteins but not to package or transmit the RNAs encoding these functions. These packaging cell lines contain the replication defective retroviral vector genome of interest, and expression of the trans-acting proteins permits the production of packaged retroviral vectors.

For the construction of packaging cell lines suitable for use in the present invention, the trans-acting viral proteins may be provided in a transient or inducible manner. Trans-acting viral genes may be placed under the control of an inducible promoter such as the tetracycline-responsive promoter (Gossen and Bujard, Proc. Natl. Acad. Sci. USA 89:5547-5551 (1992) and Pescini et al., Biochem. Biophys. Res. Comm. 202:1664-1667 (1994)). For packaging cell lines containing trans-acting genes under the control of regulated promoters, packaging can be initiated by inducing the promoter.

Cells suitable for use in preparing packaging cell lines of the present invention are derived from vertebrates and include, for example, sheep, goat, bovine, avian, primate, porcine, human, canine and the like. Typical cells for preparing packaging cells include NIH 3T3 cells (ATCC CCL-92), and derivatives thereof, however, other suitable cells are readily available.

The JSRV packaging cells are produced by introducing DNA constructs which direct the expression of trans-acting Gag, Pol and JSRV envelope (ENV) proteins that are required for packaging recombinant replication defective retroviral particles. Methods for introducing such constructs include, for example, calcium phosphate precipitation (Wigler et al., Cell 14:725 (1978); Corsaro and Pearson, Somatic Cell Genetics 7:603 (1981); Graham and Van der Eb, Virology 52:456 (1973); lipofection (Felgner et al., Proc. Natl. Acad. Sci. USA 84:7413-7417 (1987)), microinjection and electroporation (Neumann et al., EMBO J. 1:841-845 (1982), each incorporated herein by reference). The cells can also be transduced with virus, such as SV40, CMV and the like. In the case of viral vectors, cloned DNA molecules may be introduced by infection of susceptible cells with viral particles. The gag and pol genes may be derived from a wide variety of retroviruses. Retroviruses from which Gag and Pol proteins useful in the present invention can be derived from include, but are not limited to, type B, mammalian and avian type C, and type D retroviruses. For example, the gag and pol genes encoding the Gag and Pol proteins of the retroviral particles can be from, but not limited to, oncoretroviruses including, but not limited to, type B retroviruses such as murine mammary tumor virus (MMV), mammalian and avian type C retroviruses such as rous sarcoma virus (RSV), gibbon-ape leukemia virus (GALV), feline leukemia virus (FLV), Moloney murine leukemia virus (MoMLV), and type D retroviruses such as Jaagsiekte sheep retrovirus (JSRV), simian retrovirus (SRV), squirrel Monkey retrovirus (SMRV), enzootic nasal tumor virus (ENTV), and Simian Mason-Pfizer virus (MPMV) or lentivirus including, but not limited to, human immunodeficiency virus (HIV), simian immunodeficiency virus (SIV), equine infectious anemia virus (EIAV), feline immunodeficiency virus (FIV), bovine immunodeficiency virus (BIV), caprine arthritis encephalitis virus (CAEV) and Visna lentivirus (VILV). For example, the Env protein of a specific embodiment of the present invention is the JSRV Env protein and the Gag and Pol proteins are from the Moloney murine leukemia virus., and within a particular embodiment of the present invention the gag and pol genes are derived from Moloney murine leukemia virus (MoMLV), human immunodeficiency virus (HIV) or simian immunodeficiency virus (SIV) and the like.

In an alternative embodiment of the present invention, methods are provided for producing packaging cells which comprise stable and transiently expressed vector products. In one embodiment, a stable cell line is produced which expresses retroviral Gag and Pol genes. This stable cell line can be transformed transiently with a vector which encodes JSRV env and a vector which encodes a heterologous gene and packaging signal. In another embodiment a stable cell line can be constructed which expresses JSRV Env. This stable cell line can be transformed with one or more vectors which encode Gag and Pol, for example, but not limited to retroviral Gag and Pol, and a vector which encodes a heterologous gene and a retroviral packaging signal. A specific embodiment of this aspect of the invention recombinant replication-defective virus particles are made by transducing or transfecting a vertebrate cell with a first vector comprising a retrovirus gag gene; a second vector comprising a retrovirus pol gene; a third vector comprising a JSRV env gene, or a fragment thereof encoding a receptor binding fragment; and a replication defective virus particle which comprises virus RNA transcribed from a recombinant DNA provirus, the provirus comprising virus long terminal repeat sequences (LTRs), a retrovirus packaging sequence, and a heterologous gene, or a vector comprising said provirus, wherein the cell is capable of expressing and assembling retroviral proteins.

The transfected or transformed cell transiently expresses the proteins encoded by the JSRV env gene, or a fragment of the Env protein capable of binding to the JSRV endogenous receptor on a target cell, and also expresses the retrovirus Gag and Pol proteins as well as the product encoded by the heterologous gene. When expressed, a replication-defective retroviral vector particle is produced which can bind to JSRV receptors of target cells. Transient packaging can also be accomplished using a first vector which contains both the retrovirus gag and pol genes in place of the first and second vectors described above. (See, for example, Bums et al., Proc. Natl. Acad. Sci. USA 90:8033-8037 (1993); Naviux et al., J. Virol. 70:5701-5705 (1996); and Soneska et al., Nuc. Acid. Res. 23:628-633 (1995)). Other methods for transiently producing replication defective virus particles are well known to the skilled artisan. The replication defective virus particles produced transiently or by the packaging cell lines of the present invention all are capable of binding to JSRV receptors on target cells and of providing a means for the transfer of a wide variety of heterologous nucleic acid segments.

Using the JSRV packaging cell lines of the present invention, replication defective retroviral vectors are assembled into corresponding retroviral particles by surrounding the recombinant viral RNA with the Gag and Pol proteins to form a core particle and encapsulating the core particle in a membrane containing the JSRV Env protein. Thus, a packaging cell of the present invention provides replication defective retroviral particles capable of transducing cells (i.e., an infectious virus having a ribonucleoprotein core particle surrounded by a membrane containing JSRV envelope protein, or fragments thereof which are capable of binding to the JSRV receptor on target cells) containing the vectors as described herein.

The production of undesirable helper virus can be detected in a variety of ways, including, e.g., vector rescue assays in which cells containing but not producing a selectable replication-defective viral vector are transduced with the test virus and assayed for production of the vector. Rescue of the vector can be detected by passaging the cells to allow virus spread and assaying medium exposed to these cells for the selectable viral vector in a standard colony assay. Representative assays include the S⁺L⁻ assay described by Bassin et al. (Nature 229:564-566 (1971), incorporated herein by reference) and marker rescue described by Miller et al. (Meth. Enzymol. 217:581-599 (1993), incorporated herein by reference).

The replication defective retroviral vectors packaged by the JSRV packaging cell lines of the present invention provides a means for gene transfer in a wide range of animal species, including, e.g., experimental and domestic animals, livestock (e.g., sheep, goats, cows, horses), birds (e.g., chickens), cats, dogs, monkeys and primates (e.g., chimpanzees, macaques, monkeys, and humans). For livestock uses, for example, the particles produced according to the invention are useful for infecting cells in preimplantation embryos, which embryos when implanted in an animal creates a transgenic animal or an animal which expresses a gene product that it would normally not produce.

The replication defective retroviral particles packaged in a JSRV packaging cell line of the invention are used to infect (transduce) target cells, such as, for example, those which are defective in expression of the gene of interest, or which can act to secrete the desired protein. By transduction is meant the process by which non-viral genes are transferred and expressed in a host cell by a viral vector. Transduction can take place ex vivo or in vivo. When the transduction is performed ex vivo, typically the targeted cells, e.g. lung epithelial cells, lymphocytes, bone marrow, hematopoietic cells and hematopoietic stem cells, fibroblasts, hepatocytes, endothelial cells, benign or malignant tumor cells, and the like, are autologous in that they have been removed from the individual in need of the gene product of interest, but allogeneic or even xenogeneic cells may also be employed. The cells are infected by the replication defective virus particles containing the gene of interest, and the cells are returned (or transplanted) to the host. When the transduction of the host cells is ex vivo, typically medium containing the recombinant replication defective virus particles is incubated with the target cells. The target cells may be cultivated ex vivo to expand their numbers in primary cell cultures. Transduction is typically during the early days of host cell culture, and may be accomplished by co-cultivating the target cells with a cell line producing replication defective virus vectors. The target cells are not necessarily cultured prior to transduction and replacement in the host patient.

For in vivo transduction, the replication defective virus particles can be administered to a host in a wide variety of ways. The particular mode of administration will depend upon several factors, including, among others, the particular use intended, the host being treated, the tissue targeted for transduction, the gene product of interest, the general health of the host, i.e., a patient, and the like, but will generally be administered intradermally, subcutaneously, intramuscularly, topically (e.g., aerosol, such as via a nebulizer), intravenously, intraperitoneally, and the like. Thus, the vectors can be administered to tissues and organs (e.g., via a catheter) such as the lungs, bladder, urethra, uterus, liver, heart and circulatory system, kidney, bone marrow, brain, lymphoid tissues, stomach, small intestine, large intestine, colon, prostate, and the like.

The dosages of the replication defective virus vectors produced according to the invention will be determined through empirical experiments such as dose escalation studies and the like. It must be kept in mind that the materials of the present invention may be employed in serious disease states, that is, life-threatening or potentially life threatening situations. In such cases, in view of the ability of the replication defective virus particles produced by the present invention to infect a wide variety of vertebrate cells, it is possible and may be felt desirable by the treating physician to administer substantial excesses of these viral particles.

In a further embodiment the present invention provides chromosomal localization of a potential human receptor for the Jaagsiekte sheep retrovirus (JVR) to the p21.3 region of human chromosome 3. The majority of retroviral receptors do not localize to chromosome 3, but most of the CC-chemokine receptor genes (CCRs) which have been identified as co-receptors for lentiviruses have been demonstrated to map within the 3p21.3 region. The present inventors have demonstrated that JVR does not map to the same regions as previously defined for retroviruses, lentiviruses, or CC-chemokine receptors and is a new retroviral receptor in human cells.

The following examples are offered by way of illustration, not by way of limitation.

### EXAMPLE 1

This example describes the construction of hybrid retrovirus packaging cells that express JSRV Env and the MoMLV Gag-Pol proteins. These packaging cells were able to produce JSRV-pseudotype vectors at high titer. These replication-defective viral particles were used to determine the host range of JSRV Env and to determine a JSRV receptor on certain mammalian cells including human cells. The human receptor was localized to a gene within the p21.3 region of human chromosome 3.

Cell culture. Mammalian cells including SSF-123 primary sheep skin fibroblasts, HT-1080 human fibrosarcoma cells (ATCC CCL-121), 293 human kidney epithelial cells (ATCC CRL 1573), 1B3 immortalized human bronchial epithelial cells (Zeitlin et al., Am. J. Respir. Cell Mol. Biol. 4:313-319 (1991)), HeLa cervical carcinoma cells (ATCC CCL-2), NIH 3T3 thymidine kinase-deficient mouse embryo fibroblasts (Wei et al., J. Virol. 39:935-944 (1981)), M. dunni tail fibroblasts (Chattopadhyay et al., Virology 113:465-483 (1981)), D17 canine osteosarcoma cells (ATCC CRL-6248), 208F rat embryo fibroblasts (Quade et al., Virology 98:461 -465 (1979)), MDBK bovine kidney epithelial cells (ATCC CCL-22), Vero African green monkey kidney epithelial cells (ATCC CCL-81), MF-NAN primary mouse (BALB/c) fibroblasts, MF-H1 primary mouse (C57BL/6) fibroblasts, and RbTE rabbit tracheal epithelial cells were grown in DMEM supplemented with 10% fetal bovine serum (Hyclone). RbTE cells were immortalized by transduction with human papilloma virus E6 and E7 genes in a retrovirus vector, LXSN16E6E7 (Halbert et al., J. Virol. 65:473-478 (1991)). G355 feline embryonic brain cells (ATCC CRL-2033; Dunn et al., J. Virol. 67:4704-4711 (1993)) were grown in McCoy's medium supplemented with 15% fetal bovine serum. Chinese hamster ovary cells (CHO, ATCC CCL-61), A23 hamster cells and the A23 derived radiation hybrid clones (Stewart et al., Genome Res. 7:422-433 (1997)), were grown in minimal essential medium-alpha supplemented with 10% fetal bovine serum.

Retroviral vectors and virus titer. Nomenclature for retroviral vectors and pseudotypes has been discussed before (Miller et al., J. Virol. 71:4531-4535 (1997)). LAPSN is an MoMLV-based vector encoding the human placental alkaline phosphatase (AP) and the neomycin phosphotransferase genes (Miller et al., Proc. Natl. Acad. Sci. USA 91:78-82 (1994)). Vectors with a JSRV pseudotype were made by using the pSX2.Jenv plasmid (FIG. 1), which was constructed by inserting the 1883 bp MslI to Ecl136 fragment of JSRV-_{JS7} containing the Env coding region into the BsaAI- and MscI-cut 4,239-bp backbone of the pSX2 plasmid (Miller et al., J. Virol. 70:5564-5571 (1996)) by using blunt-end ligation.

JSRV-_{JS7} is a proviral clone derived from a λ phage library of genomic DNA from the JS7 cell line that was derived from a spontaneous case of OPC. Retrovirus vector titers were determined as described previously, either by assaying for alkaline phosphatase positive (AP⁺) focus-forming units (FFU) (Fields-Berry et al., Proc. Natl. Acad. Sci. USA. 89:693-697 (1992)), or G418-resistant colony forming units (CFU) (Miller et al., Biotechniques 7:980-990 (1989)).

Generation and analysis of JSRV-pseudotype retrovirus packaging cells. Stable retrovirus packaging cells expressing JSRV Env were generated by cotransfection (Miller et al., Biotechniques 7:980-990 (1989)) of pSX2.Jenv plasmid DNA along with a 20 hygromycin phosphotransferase (hpt) gene contained in plasmid pSV2Δ13-hyg into LGPS cells, selection in hygromycin, and isolation and screening of individual clones (PJ1 through PJ24) as previously described (Miller et al., J. Virol. 70:5564-5571 (1996)). Briefly, NIH 3T3 cells that can express the MoMLV Gag-Pol proteins (LGPS cells, Miller et al., J. Virol. 65:2220-2224 (1991)) were transfected with plasmid pSX2.Jenv and a plasmid encoding hygromycin prophotransferase (pSV2Δ13-hyg) at a 20:1 or 100:1 ratio. Resistant clones were isolated and the cloned cell lines were tested for their ability to produce virus by marker rescue.. The packaging cells which gave the highest titer were transduced with LAPSN(PT67) vector (Miller et al., J. Virol. 70:5564-5571 (1996)) to generate stable LAPSN producer lines producing JSRV-pseudotype LAPSN vector (LAPSN(PJ)) free of replication-competent virus.

Marker rescue assay. SSF and HT-1080 cells were plated at 10⁵ cells per 6-cm dish on day 1, were transduced with LAPSN(PT67) virus at an multiplicity of infection (MOI) of approximately 1 in the presence of Polybrene (4 μg/ml) on day 2, and were trypsinized and replated in G418 (active concentration of 250 μg/ml for SSF and 400 μg/ml for HT-1080 cells). These polyclonal populations of cells carrying the LAPSN vector were used in the marker rescue assay for helper virus as described (Miller et al., J. Virol. 70:5564-5571 (1996)). Briefly, the SSF/LAPSN or HT-1080/LAPSN cells were plated at 5×10⁵ cells per 6-cm dish on day 1, were infected with 0.5 ml of LAPSN(PJ) test virus (6×10⁵ AP FFU/ml) in the presence of Polybrene on day 2, and were trypsinized and split 1:10 every 2 to 3 days for 2 weeks while being kept at high density to facilitate potential virus spread. After 2 weeks, medium harvested from confluent dishes of respective cells were tested for LAPSN vector rescue and transfer using SSF cells as targets. MoMLV was used as a positive control to show that helper virus could rescue the LAPSN vector in these cells. The passaged SSF/LAPSN and HT-1080/LAPSN cells were also stained for alkaline phosphatase (AP) to ensure that they still retained the LAPSN vector.

Radiation Hybrid mapping of Jaagsiekte Sheep Retrovirus Receptor and STRL33 genes. The Stanford G3 panel of human whole genome hybrid cell lines was used for phenotypic Radiation Hybrid (RH) mapping (www.shgc.stanford.edu/ Mapping/rhi/) (Stewart et al., Genome Research 7:422-433 (1997)). For mapping Jaagsiekte Sheep Retrovirus receptor (JVR), the RH cell lines were plated at 5×10⁴ cells per well in a 6-well plate and exposed to LAPSN(JSRV) vector the next day. Alkaline phosphatase assays were performed as explained above, and AP⁺FFU were counted to measure transduction.

To map the STRL33 gene, the Stanford G3 panel RH DNA (Research Genetics, Huntsville, Ala.) was used for genotypic mapping. PCR was performed with the STRL33-specific primers 5′-GCCAGGGTTTCGAGAAGCTGCTCTGGAATT-3′(SEQ ID NO: 5) and 5′-TCATAGTC CCTGGTGCTAGTTATTCTGGAT-3′(SEQ ID NO: 6). Genomic DNA samples from A3 hamster cell line (Stewart et al., supra, (1997)) and RM human lymphoblastoid cell line (Stewart et al., supra, (1997)) were used as negative and positive controls, respectively. All PCR amplifications were performed as follows: Initial denaturation at 94° C. for 2 min. followed by 35 cycles of amplification at 94° C. for 30 sec. at 62° C. for 1 min and at 68° C. for 4 min. with a final extension at 68° C. for 10 min. The PCR products were electrophoresed on 1% agarose gels and visualized by ethidium bromide staining.

### Results

The JSRV Env protein can pseudotype an MoMLV-based retrovirus vector and promote transduction of human cells. The ability of JSRV Env to recognize the MoMLV Gag-Pol proteins and to rescue the MoMLV-based LAPSN vector by transient transfection of a JSRV Env expression plasmid (pSX2.Jenv) into LGPS/LAPSN cells was tested. LGPS/LAPSN cells contain the LAPSN vector and contain the pLGPS plasmid for expression of MoMLV Gag-Pol proteins (Miller et al., J. Virol. 70:5564-5571 (1996)). Transfection of the pSX2 plasmid DNA expressing the 10A1 murine leukemia virus Env (Miller et al., J. Virol. 70:5564-5571 (1996)) was used as positive control. Two days after transfection, the medium from these cells was harvested and assayed for vector production using SSF-123 sheep skin fibroblasts and HT-1080 human fibrosarcoma cells as targets for transduction (Table 1).

TABLE 1
Transduction of sheep and human cells by the LAPSN vector
with a JSRV, 10A1, or xenotropic pseudotype^{a}

| | |
|---|---|
| LAPSN | LAPSN titer (FFU/ml) measured on: |

| pseudotype | SSF | HT-1080 |
|---|---|---|
| None | <1 | <1 |
| JSRV | 2 × 10⁴ | 2 × 10³ |
| 10A1 | 80 | 2 × 10³ |
| Xenotropic | 1 × 10³ | 1 × 10⁴ |

^{a}JSRV and 10A1 MLV pseudotypes of the LAPSN vectors were made by calcium phosphate-mediated transient transfection (Miller et al., Biotechniques 7:980-990 (1989)) of LGPS/LAPSN cells with the JSRV or 10A1 env construct. As a negative control, Env LAPSN vector (no pseudotype) were generated by transient transfection of LGPS/LAPSN cells with a plasmid that did not contain an env gene. Xenotropic-pseudotype vector was made by using a packaging cell line containing the LAPSN vector and MoMLV gag-pol
# and env genes. Vector titers were measured on the indicated target cells, and results are means of at least two experiments.

Both cell types could be transduced by the JSRV-pseudotype LAPSN vector, demonstrating the ability of JSRV Env to pseudotype MoMLV based vectors packaged with MoMLV Gag proteins as well as the ability of JSRV Env to promote entry into human cells. The JSRV-pseudotype vector titer was about 10-fold higher on the sheep than on the human cells. In contrast, the titer of the 10 A1- or xenotropic-pseudotype LAPSN vectors was at least 10-fold higher on the human than on the sheep cells, showing the preference of the JSRV-pseudotype vector for sheep cells. These results indicated the potential to develop a JSRV pseudotype packaging line capable of producing high-titer virus capable of transducing human cells.

High titer vector production in the absence of replication-competent virus. Stable JSRV-pseudotype retrovirus packaging lines expressing JSRV Env and MoMLV Gag-Pol proteins were generated as described above, and individual clones were screened for packaging function. To show that these packaging cells could be used to generate stable-vector producing cell lines, the clone which gave the highest titer (PJ 14) was plated at 10⁵ cells per 6-cm dish on day 1, was transduced at an multiplicity of infection (MOI) of about 1 with LAPSN(PT67) virus in the presence of Polybrene on day 2, and was trypsinized and replated at 1:100 or 1:500 dilutions in medium containing G418 (500 μg/ml, active) on day 3. Individual clones were selected and screened for LAPSN vector titer using SSF cells as targets. Titers as high as 6×10⁵ AP⁺FFU/ml were obtained, demonstrating that the JSRV-pseudotype packaging cells can be used for generating high-titer retrovirus vectors. Assay of LAPSN vector produced from a high titer clone by using a marker rescue assay showed that the preparations were free of replication-competent virus (<1 FFU/ml).

Host Range of JSRV Env. The JSRV-pseudotype LAPSN vector was used to measure the ability of JSRV Env to promote transduction of a variety of mammalian cells (Table 2). The JSRV-pseudotype vector transduced various human cells, but at titers approximately 10-fold lower than that obtained using SSF-123 cells. Although the JSRV pseudotype vector transduced various human cell lines, including 1B3 bronchial epithelial cells, HT-1080 fibrosarcoma cells and 293 kidney epithelial cells, it could only transduce HeLa human cervical carcinoma cells at low levels. In addition to sheep and human cells, the vector transduced a wide range of mammalian cells including monkey, bovine, dog and rabbit cells. The vector also transduced G-355 cat cells but at very low levels. The vector was unable to transduce wild or laboratory mouse cells, rat cells or hamster cells. These rodent cells could be transduced using the LAPSN vector with a 10 A1 pseudotype, showing that the LAPSN vector and MoMLV Gag-Pol proteins are functional in these cells, as expected, and that the block to infection was at the level of virus entry mediated by the JSRV Env.

TABLE 2
Host Range of LAPSN vector produced by
JSRV-pseudotype packaging cells

| Species | Target Cells^{a} | Vector Titer (AP⁺ FFU/ml)^{b} |
|---|---|---|
| Sheep | Sheep skin fibroblasts | 6 × 10⁵ |
| Human | HT-1080 | 3 × 10⁴ |
| | IB3 | 4 × 10⁴ |
| | 293 | 4 × 10⁴ |
| | HeLa | 10 |
| Monkey | Vero | 1 × 10⁵ |
| Dog | D17 | 1 × 10⁴ |
| Bovine | MDBK | 2 × 10³ |
| Rabbit | RbTE | 2 × 10³ |
| Cat | G355 | 20 |
| Mouse | NIH 3T3 TK⁻ | <1 |
| (NIH Swiss) | | |
| Mouse | MF-NAN | <1 |
| (BALB/c) | | |
| Mouse | ME-H1 | <1 |
| (C57BL/6) | | |
| Mouse | M. dunni tail fibroblasts | <1 |
| (M. dunni) | | |
| Rat | 208F | <1 |
| Hamster | A23 | <1 |
| | CHO | <1 |

^{a}TK⁻, thymidine kinase deficient.
^{b}The LAPSN(PJ4) titer on HeLa cells was measured by production of G418-resistant colonies (CFU per milliliter) rather than AP⁺ foci because HeLa cells have high levels of endogenous heat-stable AP. Results are means of at least two experiments.

JVR maps to region p21.3 of human chromosome 3 at a site different from those of other retrovirus receptors. JSRV-pseudotype vectors transduced human but not hamster cells, indicating that it might be possible to map the position of the JSRV receptor by analyzing the susceptibility of human/hamster radiation hybrid cell lines to transduction by a JSRV vector. The G3 panel of human radiation hybrid cell lines from the Stanford Human Genome Center (SHGC) (Stewart et al., Genome Research 7:422-433 (1997)) was used for phenotypic mapping of Jaagsiekte sheep retrovirus receptor (JVR). The transduction result for the 83 ordered hybrid cell lines was 0000000000000000000000000000000000110000100 0000R0000000000000000100000000R0R00001, where 0 indicates no transduction (<10 FFU/ml), 1 indicates transduction (>100 FFU/ml), and R indicates an indeterminate result (cell clone not available for analysis). This result was submitted to the SHGC RH Server v4.0 (www.shgc.stanford.edu/RH/index.html), which mapped JVR at a distance of 18 cR_{10,000} (centiray distance for RH cells made using 10,000 rad of irradiation) from marker SHGC-11855 on chromosome 3 with a highly significant LCD score (log₁₀ of the likelihood ratio) of 6.77. Subsequently, the multiple integrated maps at the National Center for Biotechnology Information (NCBI) Entrez Genomes site (www.ncbi.nlm.nih.gov/Entrez/Genome/org.html) was used to map JVR to a position within the region p21.3 of chromosome 3.

The chromosomal locations of most other known retrovirus receptors have been determined (Table 3). Many of these map to chromosomes other than chromosome 3, showing that JSRV does not use previously determined receptors for cell entry. Careful examination of the p21.3 region of human chromosome 3 showed that none of the previously mapped retroviral receptors localize to the same position. However, the lentivirus receptor STRL33 (Bonzo, TYMSTR) had been assigned to chromosome 3, but had not been more precisely localized (Loetscher et al., Cell Biol. 7:652-660 (1997)). These results indicate that JVR is probably a new retroviral receptor, but might be the same as STRL33.

STRL33 (Bonzo) maps to 3p21.3 region, approximately 500 kb telomeric to the 285 kb Chemokine receptor (CCR) cluster. To determine whether or not STRL33 might function as the JSRV receptor, the Stanford G3 panel RH DNA was used for mapping the STRL33 gene, as described above. The PCR results for DNA samples from the 83 ordered hybrids was 000000000000100R0010000000000000001000011000000000000 0000000001000010000R001000010, where 0 indicates PCR negative, 1 indicates PCR positive, and R indicates an indeterminate result. This bar code was submitted to the SHGC RH Server v4.0, which mapped STRL33 at a distance of 13 cR_{10,000}(LOD 9.46) from marker SHGC-12886. Using the multiple integrated maps at the NCBI Entrez-Genomes site we have localized STRL33 gene to about 500 kb away from the CCR cluster in chromosome 3p21.3 and about 7.5 Mb from JVR, demonstrating that STRL33 and JVR do not localize to the same position and that JVR is not likely to be any of the known retroviral receptors.

TABLE 3
Chromosomal localization of retrovirus receptors in the human genome.

| Retrovirus | | | | |
|---|---|---|---|---|
| group | Receptor name | Localization | Method^{a} | Reference |
| Ecotropic | CAT1 (Rec1, | 13q12 | In situ hybridization, RFLP | Albritton et al., Genomics 12:430-434 (1992) |
| MLV | SLC7A1) | | | |
| GALV/ | PIT1 (GLVR1, | 2q11-q14 | In situ hybridization | Kaelbling et al., J. Virol. 65:1743-1747 (1991) |
| FeLV-B | SLC20A1 | | | |
| Amphotropic | PIT2 (Ram1, GLVR2, | 8p11-q11 | Somatic cell hybrids | Garcia et al., J. Virol. 65:6316-6319 (1991) |
| MLV | SLC20A2) | | | |
| Xenotropic | XPR1 | 1q25.1 | Radiation hybrids | Battini et al., Proc. Natl. Acad. Sci. USA. 96:1385-(1390) |
| and | | | | |
| polytropic | | | | |
| MLV | | | | |
| RD114/ | RDR (SLC1A5) | 19q13.3 | Radiation hybrids | Rasko et al., Proc. Natl. Acad. Sci. USA 96:2129-2134 (1999) |
| simian type | | | | |
| D retrovirus | | | | |
| FeLV-C | FLVCR | 1q32.1 | Radiation hybrids | unpublished data |
| T-cell | HTLVR | 17q23 | Somatic cell hybrids | Gavalchin et al., Virology 212:196-203 (1995) |
| leuKemia | | | | Tajima et al., Somat. Cell Molec. Genet. 23:225-227 (1997) |
| viruses | | | | |
| (HTLV, | | | | |
| STLV) | | | | |
| Lentivirus | CD4 | 12p12-p13 | DNA sequencing | Ansari-Lari et al., Genome Res. 6:314-326 (1996) |
| (human, | CCR1, −2b, −3, −5 | 3p21.3 | DNA sequencing, radiation | Samson et al., Genomics 36:522-526 (1996) |
| simian, and | | | hybrids | |
| feline | CCR4 | 3p22^{b} | Radiation hybrids | Samson et al., Genomics 36:522-526 (1996) |
| immunod- | CCR8 | 3p22 | Radiation hybrids | Samson et al., Eur. J. Immunol. 26:3021-3028 (1996) |
| deficiency | CXCR4 | 2q21 | In situ hybridization | Federsppiel et al., Genomics 16:707-712 (1993) |
| viruses) | CX3CR1 (V28) | 3p22 | FISH, Radiation hybrids | Combadiere et al., DNA Cell Biol. 14:673-680 (1995) |
| | GPR1 | 15q26.1 | FISH | Maho et al., Cytogenet. Cell Genet. 87:265-268 (1999) |
| | GPR15 (Bob) | 3q11.2- | FISH | Marchese et al., Genomics 23:462-465 (1996) |
| | | q13.1 | Heiber et al., Genomics 32:462-465 (1996) | |
| | ChemR23 | 12q21.2- | Radiation hybrids | Samson et al., Eur. J. Immunol. 28:1689-1700 (1998) |
| | | q21.3 | | |
| | APJ | 11q12 | FISH | O'Dowd et al., Gene 136:355-360 (1993) |
| | STRL33 (Bonzo, | 3p21.3 | Somatic cell hybrids, Radiation | Loetscher et al., Cell Biol. 7:652-660 (1997), |
| | TYMSTR) | | hybrids | data provided herein |
| JSRV | JVR | 3p21.3 | Radiation hybrids | data provided herein |

^{a}RFLP, restriction fragment length polymorphism; FISH, fluorescent in situ hybridization.
^{b}The localization of CCR4 given by the authors (3p24) was changed to 3p22 to reflect more recent localization data for flanking markers.

The ability of JSRV Env to promote infection of human cells in culture could be relevant to the epidemiology of human lung cancer, especially in regard to non-smokers exposed to sheep where OPC is endemic. Although there is no proof for JSRV involvement in human lung carcinoma, the possibility of viral etiology can not be excluded because of the similarity of BAC with OPC and the multifocal/multiclonal nature of some BAC cases (Palmarini et al., Trends Microbiol. 5:478-483 (1997)). Several factors could explain the absence of evidence for human infection with JSRV, such as lack of immunological reagents to detect human infections. There has been no report of any serological study to evaluate human sera for JSRV antibodies. Alternatively, the JSRV Env might be able to bind to receptors and mediate entry of the viral genome, but some of the viral replicative elements may not be functional in human cells, resulting in post-entry or replication blocks. It is known that the JSRV Gag-Pol proteins are functional during viral assembly in human cells, as evidenced by the use of an infectious molecular clone of JSRV to produce the virus in 293 human epithelial cells (Palmarini et al., J. Virol. 73:6964-6972 (1999)). However, their functionality during reverse transcription and integration of viral DNA in human cells is unknown. Another important factor might be the presence of transcriptionally active ESRV sequences in sheep genome, which may induce tolerance to JSRV antigens in sheep and allow the virus to propagate and establish an infection. On the contrary, humans and other animals may develop a strong immune response leading to virus clearance.

Chromosome localization has provided an alternative approach to interference analysis to determine retroviral receptor usage, especially in the case of viruses that do not show strong interference even to infection by the same virus. The inability of JSRV Env to promote infection of hamster cells has been used to probe a panel of human-hamster whole genome radiation hybrids to localize the JSRV receptor (JVR) gene to p21.3 region of the human chromosome 3. Although majority of the known retroviral receptors do not localize to chromosome 3, most of the CC-chemokine receptor genes (CCRs) which have been identified as coreceptors for lentiviruses have been shown to map within 3p21.3 region (Samson et al., Genomics 36:522-526 (1996)). Careful analysis of the mapping data has revealed that JVR does not map to the same positions as most of these receptors, being approximately 7 Mb away from the 285 kb cluster of CCR3, 1, 2, 5, and 6, and farther away from CCR4, CCR8, and CCR10.

The lentivirus receptor CX3CR1 has recently been mapped to 3p24 region which left one other lentivirus receptor STRL33 (Bonzo) in the same region of the JVR gene (Loetscher et al., Cell Biol. 7:652-660 (1997)). The G3 panel of RH DNA has been used to localize the STRL33 gene 500 kb telomeric to the CCR cluster in region 3p21.3 and about 7.5 Mb away from JVR. These results indicate that JVR is a new retroviral receptor in human cells.

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted.

What is claimed is:

1. A cultured packaging cell for producing a replication-defective retroviral vector particle which binds to a Jaagsiekte sheep retrovirus (JSRV) receptor, wherein the packaging cell is a vertebrate cell which can express and assemble retroviral proteins, comprising:
a first vector encoding a retroviral envelope protein having the amino acid sequence of Jaagsiekte sheep retrovirus (JSRV), or a chimeric retroviral envelope protein comprising a receptor binding fragment of JSRV envelope protein that directs the binding of the retroviral vector particle to a Jaagsiekte sheep retrovirus (JSRV) receptor on a target cell; and
a second vector comprising nucleotide sequence encoding retrovirus Gag and Pol proteins, wherein expression of the Env, Gag and Pol proteins in the packaging cell in the presence of a third vector encoding a heterologous protein of interest flanked by retrovirus packaging sequences, produces the replication-defective retroviral particle that binds to Jaagsiekte sheep retrovirus (JSRV) receptors of the target cell.

2. The cultured packaging cell of claim 1, wherein the first vector comprises a nucleotide sequence which encodes the amino acid sequence as depicted in SEQ ID NO: 2 or SEQ ID NO: 4.

3. The cultured packaging cell of claim 2, wherein the first vector comprises a nucleotide sequence as depicted in SEQ ID NO: 1 or SEQ ID NO: 3.

4. The cultured packaging cell of claim 1, wherein the Jaagsiekte sheep retrovirus (JSRV) envelope protein is a chimeric protein comprising the signal sequence, receptor binding domain and transmembrane domain of JSRV and contiguous amino acid residues of the cytoplasmic domain from a different retrovirus envelope protein.

5. The cultured packaging cell of claim 4, wherein the cytoplasmic domain is from a oncoretrovirus or a lentivirus.

6. The cultured packaging cell of claim 5, wherein the cytoplasmic domain is from MOMLV, HIV or SIV.

7. The cultured packaging cell of claim 1, wherein the replication-defective retroviral vector particle is a hybrid retroviral particle.

8. The cultured packaging cell of claim 1, wherein the retroviral Gag and Pol proteins are from an oncoretrovirus or a lentivirus.

9. The cultured packaging cell of claim 8, wherein the retroviral Gag and Pol proteins are from a type B, a mammalian and avian type C, or a type D retrovirus.

10. The packaging cell line of claim 8, wherein the Gag and Pol proteins are from a murine mammary tumor virus (MMV), a rous sarcoma virus (RSV), a gibbon-ape leukemia virus (GALV), a feline leukemia virus (FLV), a Moloney murine leukemia virus (MoMLV), a Jaagsiekte sheep retrovirus (JSRV), a simian retrovirus (SRV), a squirrel Monkey retrovirus (SMRV), an enzootic nasal tumor virus (ENTV), a Simian Mason-Pfizer virus (PMV), a human immunodeficiency virus (HIV), a simian immunodeficiency virus (SIV), an equine infectious anemia virus (EIAV), a feline immunodeficiency virus (FIV), a bovine immunodeficiency virus (BIV), a caprine arthritis encephalitis virus (CAEV) or a Visna lentivirus (VILV).

11. The cultured packaging cell of claim 1, wherein the replication-defective retroviral particle that binds to Jaagsiekte sheep retrovirus (JSRV) receptors of the target cell is produced transiently.

12. The cultured packaging cell of claim 1, wherein the replication defective retroviral particle that binds to Jaagsiekte sheep retrovirus (JSRV) receptor of the target cell is stably produced.

13. The cultured packaging cell of claim 1, wherein the heterologous nucleotide sequence encodes a protein, a peptide, or an RNA molecule.

14. The cultured packaging cell of claim 13, wherein the RNA molecule is an antisense RNA or a ribozyme.

15. The cultured packaging cell of claim 1, wherein the vertebrate cell is an avian or mammalian cell which can express and assemble retroviral proteins.

16. The cultured packaging cell of claim 1, wherein the first vector comprises a nucleotide sequence encoding the Jaagsiekte sheep retrovirus (JSRV) envelope protein, or comprises a nucleotide sequence encoding the JSRV receptor binding fragment thereof, and the second vector comprises a nucleotide sequence encoding the retrovirus Gag and Pol proteins are integrated in a chromosome of the packaging cell.

17. A cultured packaging cell for producing a replication-defective retroviral vector particle, wherein the packaging cell is a vertebrate cell which can express and assemble retroviral proteins, comprising:
a first vector comprising a Jaagsiekte sheep retrovirus (JSRV) envelope gene encoding an amino acid residue sequence as depicted in SEQ ID NO: 2 or 4, or a Jaagsiekte sheep retrovirus (JSRV) receptor binding fragment thereof, that directs the binding of the retroviral vector particle to Jaagsiekte sheep retrovirus (JSRV) receptors on a target cell;
a second vector encoding a retrovirus Gag and Pol protein; and
a third vector having a sequence comprising a heterologous gene of interest flanked by retrovirus packaging signal sequences, wherein upon expression of the Env, Gag and Pol proteins in the packaging cell a replication-defective retroviral vector particle is produced that binds to Jaagsiekte sheep retrovirus (JSRV) receptors of the target cell.

18. A cultured packaging cell line for producing a replication-defective retroviral vector particle which binds to a Jaagsiekte sheep retrovirus (JSRV) receptor, wherein the packaging cell is a vertebrate cell which can express and assemble retroviral proteins, comprising:
a first vector encoding a retroviral envelope protein having the amino acid sequence of a Jaagsiekte sheep retrovirus (JSRV) envelope protein, or a receptor binding fragment thereof, that directs the binding of the retroviral vector particle to Jaagsiekte sheep retrovirus (JSRV) retroviral receptors on a target cell;
a second vector encoding a retrovirus Gag and Pol protein; and
a third vector comprising a nucleic acid sequence encoding a heterologous gene of interest flanked by retrovirus packaging signals, wherein upon expression of the Env, Gag and Pol proteins in the packaging cell in the presence of the third vector having the sequence of the heterologous gene of interest, produces the replication-defective retroviral particle that bind to Jaagsiekte sheep retrovirus (JSRV) receptors of the target cell.

19. A method for producing a replication-defective retroviral vector particle comprising a heterologous gene of interest, comprising:
transducing or transfecting a retroviral packaging cell with
(a) a replication defective virus particle which comprises virus RNA transcribed from a recombinant DNA provirus, said provirus comprising virus long terminal repeat sequences (LTRs), a retrovirus packaging sequence, and a heterologous gene,or
(b) a vector comprising said provirus, wherein said packaging cell is a vertebrate cell which is capable of expressing and assembling retroviral proteins and having (i) an integrated vector encoding a Jaagsiekte sheep retrovirus (JSRV) envelope protein having amino acid residue sequence of SEQ ID NO: 2 or 4, or a fragment thereof, that direct binding of the retroviral particle to Jaagsiekte sheep retrovirus (JSRV) receptors on a target cell, and (ii) an integrated vector encoding retrovirus Gag and Pol proteins;
expressing in said packaging cell the sequences which encode the Jaagsiekte sheep retrovirus (JSRV) Env protein, the retrovirus Gag and Pol proteins, and the sequence that comprises the heterologous gene of interest; and
producing a replication-defective retroviral vector particle that binds to Jaagsiekte sheep retrovirus (JSRV) receptors of human target cells.

20. The method of claim 19, wherein the replication-defective retroviral vector particle containing the heterologous gene of interest is produced at a titer of up to 10⁶ FFU/ml medium.

21. The method of claim 19, wherein the replication-defective retroviral vector particle containing the heterologous gene of interest is produced at a titer of at least 5×10⁶ FFU/ml medium.

22. The replication-defective retroviral vector particle containing the heterologous gene of interest produced by the method of claim 19.

23. A method for producing a replication-defective retroviral particle comprising a heterologous gene of interest, comprising:
transducing or transfecting human target cells with (i) a first vector comprising a retrovirus gag gene; (ii) a second vector comprising a retrovirus pol gene; (iii) a third vector comprising a JSRV env gene; and (iv) (a) a replication defective virus particle which comprises RNA transcribed from a recombinant DNA provirus, the provirus comprising virus long terminal repeat sequences (LTRs), a retrovirus packaging sequence, and a heterologous gene, or (b) a vector comprising the provirus, wherein the cell is capable of expressing and assembling retroviral proteins;
expressing in the cell the products encoded by the Jaagsiekte sheep retrovirus (JSRV) env gene, the retrovirus gag and pol genes, and the sequence that comprises the heterologous gene of interest; and
producing transiently a replication-defective retroviral vector particle that binds to Jaagsiekte sheep retrovirus (JSRV) receptors of human target cells.

24. A method for producing a replication-defective retroviral particle comprising a heterologous gene of interest, comprising:
transducing or transfecting human target cells with (i) a first vector comprising a retrovirus gag and pol genes (ii) a second vector comprising a Jaagsiekte sheep retrovirus (JSRV) env gene; and (iii) (a) a replication defective virus particle which comprises RNA transcribed from a recombinant DNA provirus, said provirus comprising virus long terminal repeat sequences (LTRs), a retrovirus packaging sequence, and a heterologous gene, or (b) a vector comprising said provirus, wherein the target cell is capable of expressing and assembling retroviral proteins;
expressing in the target cell the products encoded by the Jaagsiekte sheep retrovirus (JSRV) env gene, the retrovirus gag and pol genes, and the sequence that comprises the heterologous gene of interest; and
producing transiently a replication-defective retroviral vector particle that binds to Jaagsiekte sheep retrovirus (JSRV) receptors of human target cells.


SEQUENCE LISTING (6 sequences)

SEQ ID NO: 1 - DNA, 1849 bp, Jaagsiekte Sheep Retrovirus
atgccgaagcgccgcgctggattccggaaaggctggtacgcgcggcagaggaactccctgacgcatcagatgcaacgcatgaccctgagtgagcccacgagtgagctgcccacccagaggcaaattgaagcgctaatgccgtacgcctggaatgaggcacatgtacaaccccccgtgacacctactaatatactgattatgttgttgttactattacagcgggtacaaaatggggcagctgcggctttttgggcatatattcctgatccgccaatgattcaatccttaggatgggatagagaaattgtacctgtatatgttaatgatacgagccttttggggggtaaatcagatattcacatttcccctcagcaagcaaatatttctttttatggccttactactcaataccccatgtgcttttcttatcaatcgcagcatccccattgtatacaggtgtcagccgacatatcatatcctcgagtgactatttcaggcattgatgaaaaaacagggaaaaaatcgtatgggaacggaactggacccctcgatattccgttttgtgacaagcatttaagcatcggcataggcatagacactccgtggactttatgtcgagcccgggttgcatcggtgtataatatcaataatgccaatgccacctttttatgggactgggcacctggaggaacacctgattttcctgagtatcgaggacagcatccacctattttttcggtgaacaccgctcctatatatcagacggaactatggaaacttttggctgcctttggtcatggcaatagcctgtatttacagcccaatattagcggaaccaaatatggtgatgtgggagttacaggatttttatatccccgagcttgtgttccttacccattcatgttgatacaaggccatatggaaataacgctgtcattaaatatttatcatctaaactgttctaattgtatacttactaactgtattagaggagtagccaaaggagaacaagttatcatagtaaaacagcctgcttttgtaatgctgcccgttgaaatagctgaagcatggtatgacgagactgctttagaattattacaacgtattaatacggctcttagccgtcctaagagaggcttgagcctaattatcctgggtatagtatctttaatcaccctaatagctactgctgttacggcttgcgtatctttagcacaatccattcaagctgcacatacagtggattccttatcatataatgttactaaagtaatgggaactcaagaggatatagataagaaaatagaagataggttatcagctctgtatgatgtagttagagtcctaggagaacaagttcagagcattaattttcgcatgaaaatccagtgtcatgctaattataaatggatttgtgttacaaaaaagccttataatacttctgattttccatgggataaagtaaaaaaacatttgcaaggaatttggttcaatactaatctatcgttagatcttttacaactacataatgaaattcttgacattgagaattcgccaaaggctaccttaaatatagccgataccgtcgataatttcttgcaaaatttattttctaatttccctagtcttcattcattgtggaaaaccctgatcggtctaggaatatttgtgataattattgctatcgtaatctttgtatttccctgtgtcgttcgtggtctcgttcgtgattttttaaagatgagagttgaaatgctacatatgaaatatagaactatgttacaaacaccgacatctaatggagcttttaaagaataaagagaggggagctgcgggggacgacccgtga

SEQ ID NO: 2 - protein, 615 aa, Jaagsiekte Sheep Retrovirus
MPKRRAGFRKGWYARQRNSLTHQMQRMTLSEPTSELPTQRQIEALMPYAWNEAHVQPPVTPTNILIMLLLLLQRVQNGAAAAFWAYIPDPPMIQSLGWDREIVPVYVNDTSLLGGKSDIHISPQQANISFYGLTTQYPMCFSYQSQHPHCIQVSADISYPRVTISGIDEKTGKKSYGNGTGPLDIPFCDKHLSIGIGIDTPWTLCRARVASVYNINNANATFLWDWAPGGTPDFPEYRGQHPPIFSVNTAPIYQTELWKLLAAFGHGNSLYLQPNISGTKYGDVGVTGFLYPRACVPYPFMLIQGHMEITLSLNIYHLNCSNCILTNCIRGVAKGEQVIIVKQPAFVMLPVEIAEAWYDETALELLQRINTALSRPKRGLSLIILGIVSLITLIATAVTACVSLAQSIQAAHTVDSLSYNVTKVMGTQEDIDKKIEDRLSALYDVVRVLGEQVQSINFRMKIQCHANYKWICVTKKPYNTSDFPWDKVKKHLQGIWFNTNLSLDLLQLHNEILDIENSPKATLNIADTVDNFLQNLFSNFPSLHSLWKTLIGLGIFVIIIAIVIFVFPCVVRGLVRDFLKMRVEMLHMKYRTMLQHRHLMELLKNKERGAAGDDP

SEQ ID NO: 3 - DNA, 1848 bp, Jaagsiekte Sheep Retrovirus
atgccgaagcgccgcgctggattccggaaaggctggtacgcgcggcagaggaactccctgacgcatcaaatgcaacgcatgacgctgagcgagcccacgagtgagctgcccacccagaggcaaattgaagcgctaatgcgctacgcctggaatgaggcacatgtacaacctccggtgacacctactaacatcttgatcatgttattattattgttacagcgggtacaaaatggggcagctgcggctttttgggcgtacattcctgatccgccaatgattcaatccttaggatgggatagagaaatagtacccgtatatgttaatgatacgagccttttaggaggaaaatcagatattcacatttcccctcagcaagcaaatatctctttttatggccttaccactcaatatcccatgtgcttttcttatcaatcgcagcatcctcattgtatacaggtatcagctgacatatcatatcctcgagtgactatctcaggcattgatgaaaaaactgggaaaaaatcatacgggaacggatctggacccctcgacattccgttttgtgacaagcatttaagcattggcataggcatagacactccttggactttatgtcgagcccgggtcgcgtcagtatataacatcaataatgccaatgccacctttttatgggattgggcacctggaggaacacctgattttcctgaatatcgaggacagcatccgcctattttctctgtgaataccgctccaatataccaaacggaactatggaaacttttggctgcttttggtcatggcaatagtttatatttacagcccaatatcagtggaagcaaatatggtgatgtaggagttacaggatttttatatcctcgagcttgcgtgccgtatccattcatgttgatacaaggccatatggaaataacactgtcattaaatatttatcatttgaattgttctaattgcatactgactaattgtatcaggggagtagccaaaggagaacaggttataatagtaaaacagcctgcctttgtaatgctgcccgttgaaatagctgaagcctggtatgatgaaactgctttagaattattacaacgcattaatacggctcttagccgccctaagagaggcctgagcctgattattctgggtatagtatctttaatcaccctcatagctacagctgttacggcttccgtatctttagcacagtctattcaagctgcgcacacggtagactccttatcatataatgttactaaagtgatggggacccaagaagatattgataaaaaaatagaagataggctatcagctctatatgatgtagtcagagtcttaggagagcaagttcagagcattaattttcgcatgaaaatccaatgtcatgctaactataaatggatttgtgttacaaaaaaaccatacaacacttctgattttccatgggacaaagtgaagaaacatttgcaaggaatttggttcaatactaatctatcgttagaccttttacaactgcataatgagattcttgatattgaaaattcgccgaaggctacactaaatatagccgatactgttgataatttcttgcaaaatttattctctaatttccctagtctccattcgctgtggaaaaccctgattggtgtaggaatacttgtgtttattataattgtcgtaatccttatatttccttgcctcgttcgtggcatggttcgcgactttctaaagatgagagttgaaatgctgcatatgaaatatagaaatatgttacagcaccaacatcttatggagcttttaaaaaataaagagaggggagatgcgggggacgacccgtga

SEQ ID NO: 4 - protein, 615 aa, Jaagsiekte Sheep Retrovirus
MPKRRAGFRKGWYARQRNSLTHQMQRMTLSEPTSELPTQRQIEALMRYAWNEAHVQPPVTPTNILIMLLLLLQRVQNGAAAAFWAYIPDPPMIQSLGWDREIVPVYVNDTSLLGGKSDIHISPQQANISFYGLTTQYPMCFSYQSQHPHCIQVSADISYPRVTISGIDEKTGKKSYGNGSGPLDIPFCDKHLSIGIGIDTPWTLCRARVASVYNINNANATFLWDWAPGGTPDFPEYRGQHPPIFSVNTAPIYQTELWKLLAAFGHGNSLYLQPNISGSKYGDVGVTGFLYPRACVPYPFMLIQGHMEITLSLNIYHLNCSNCILTNCIRGVAKGEQVIIVKQPAFVMLPVEIAEAWYDETALELLQRINTALSRPKRGLSLIILGIVSLITLIATAVTASVSLAQSIQAAHTVDSLSYNVTKVMGTQEDIDKKIEDRLSALYDVVRVLGEQVQSINFRMKIQCHANYKWICVTKKPYNTSDFPWDKVKKHLQGIWFNTNLSLDLLQLHNEILDIENSPKATLNIADTVDNFLQNLFSNFPSLHSLWKTLIGVGILVFIIIVVILIFPCLVRGMVRDFLKMRVEMLHMKYRNMLQHQHLMELLKNKERGDAGDDP

SEQ ID NO: 5 - DNA, 30 bp, Artificial Sequence
  Description of Artificial Sequence  PCR Primer
gccagggtttcgagaagctgctctggaatt

SEQ ID NO: 6 - DNA, 30 bp, Artificial Sequence
  Description of Artificial Sequence  PCR Primer
tcatagtccctggtgctagttattctggat